SEQUENCE LISTING (1 sequence)

SEQ ID NO: 1 - RNA, 197 nt, Artificial
  ARTIFICAL SEQUENCE VARIANT FROM A VIRUS
gggguuuccaaccggaauuugagggaugccuaggcaucccccgugcgucccuuuacgagggauugucgacucuagucgacgucugggcgaaaaauguacgagaggaccuuuucgguacagacgguaccugagggaugccuaggcauccccgcgcgccgguuucggaccuccagugcguguuaccgcacugucgaccc

(19) United States
(12) Patent Application Publication
(10) Pub. No.: US 2006/0212947 A1
(43) Pub. Date: Sep. 21, 2006

(54) Non-naturally occurring animal that expresses an untranslated non-coding RNA

(75) Inventors: Abraham Grossman, Pleasantville, NY (US); Michael Evgenev, Moscow (RU)

Correspondence Address: DAVIDSON, DAVIDSON & KAPPEL, LLC, 485 SEVENTH AVENUE, 14TH FLOOR, NEW YORK, NY, 10018, US

(73) Assignee: Q-RNA, Inc., New York, NY (US)

(21) Appl. No.: 11/328,882

(22) Filed: Jan. 10, 2006

Related U.S. Application Data
(60) Provisional application No. 60/643,910, filed on Jan. 10, 2005.

(51) Int. Cl. A01K 67/027

(52) U.S. Cl. 800/3; 800/18

(57) ABSTRACT

Embodiments of the present invention feature compositions and methods for making an animal and an animal exhibiting characteristics of an agglomeration disease. The method comprises the steps of providing an animal cell and placing a vector in said cell having a non-coding RNA under the control of a promoter and controlling the expression of said non-coding RNA with said promoter.

This application claims priority to Provisional Application Ser. No. 60/643,910, filed Jan. 10, 2005, the disclosure of which is hereby incorporated by reference in its entirety.

### FIELD OF THE INVENTION

The present invention relates to the field of agglomeration proteins and diseases associated with such proteins. Embodiments of the present invention relate to compositions, methods, non-naturally occurring animals and model systems for determining factors capable of participating in and potentially altering agglomeration disease processes.

### BACKGROUND OF THE INVENTION

Some disease processes appear to stem from the misfolding of proteins. That is, of the alternative structural isoforms of a protein, some are not functional in the normal manner in which the protein interacts with other cellular constituents. For example, such misfolded proteins may exhibit changes in solubility in physiological fluids or susceptibility to degradation, either by enzymes or other means. The misfolded proteins can exhibit changes in resistance to proteinase-K digestion. The misfolded proteins can change folded conformations from alpha helices to beta sheets, or visa versa. The misfolded protein can bind an RNA molecule. The RNA molecule, bound to the misfolded protein can be resistant to enzyme digestion. The proteins can be full-length proteins, or fragments thereof. The conformation of the proteins can include alpha helix and/or beta sheet structures.

This misfolding is often associated with molecular facilitators, i.e., nucleic acids and binding factors. Essentially there are two types of nucleic acid found in living cells. One is deoxyribonucleic acid (“DNA”), and the other is ribonucleic acid (“RNA”). Under normal physiological conditions, both of these nucleic acid molecules are associated with proteins and form nucleoprotein complexes. These proteins can include scaffolding and chaperone proteins, enzymes, ligases, telomerases, etc. These nucleic acid binding proteins perform functions necessary for normal metabolism and cell/tissue viability.

In the absence of certain misfolding facilitators, nucleic acids and binding factors, the protein assumes a normal isoform. However, in the presence of certain nucleic acids and binding factors, the protein assumes a misfolded isoform. These misfolded proteins, under certain conditions and with the presence of molecular facilitators accumulate in vivo and form clusters or agglomerations. As used herein, the term “agglomeration” refers to a stable association of misfolded proteins. The term “agglomeration protein” refers to the proteins participating in the agglomeration process. That is, the term refers to the predominant protein of the agglomeration, the misfolded isoform.

Binding molecular factors or facilitators are participants in the agglomeration process which act as a continuous cascade. These binding factors can be other proteins. However, these binding factors may be complexes of several components. Most commonly, such binding factors are lipid and protein complexes. Although the binding factors and RNA may be found in small concentrations in the agglomeration, these constituents have a scaffolding and/or chaperone function.

The misfolded proteins can go on to form pathological agglomerations. These agglomerations have been shown to be associated with neuronal cell death and brain wasting diseases such as Alzheimer's, Huntington's and Parkinson's diseases, as well as polyglutamine-associated and tau-associated diseases in humans, scrapie, mad cow disease and chronic wasting diseases in animals. Spongiform encephalopathies, often involved with certain neuronal cell death and brain wasting syndromes, characteristically have protein plaques or agglomerations made manifest upon dissection. In spongiform encephalopathies, prion proteins are thought to be the etiologic agent. Prion-based diseases result from “infectious proteins” that are cellular benign prion proteins misfolded into an infectious isoform. This infectious prion protein isoform is involved in pathological prion protein agglomeration. Misfolded proteins also appear to damage cells in the lung, heart, kidney, pancreas and other organs. The presence of these misfolded proteins can be detecting with thioflavin (or derivatives or mixtures thereof), Congo-red staining or birefringence.

It is believed that these misfolded proteins that result in agglomeration-causing disease are associated with certain molecular facilitators, i.e., RNA molecules and other binding factors. Currently, a need exists for compositions and methods that can identify these molecular facilitators involved in protein misfolding. Identification of these molecular facilitators permits a definitive identification of pathological conditions. And, there exists a need for methods and compositions to facilitate identifying agents which interfere with the formation of the misfolded proteins and/or agglomeration complex, or alter the stability of the complex.

### SUMMARY OF THE INVENTION

Embodiments of the present invention are directed to systems, methods and transformed animals having utility to determine or identify compositions that participate in agglomeration disease processes. The compositions may suggest cofactors that accelerate the agglomeration process or suggest compositions that inhibit, prevent or destabilize agglomerations formation. As used herein, the term “participate” includes chaperone, or scaffold functions, acceleration, inhibition, prevention, stabilization or destabilization of agglomeration processes.

In certain embodiments, the present invention is directed to a non-human transgenic animal for use in the study of diseases associated with protein misfolding, wherein the transgenic animal expresses an untranslated non-coding RNA capable of binding an agglomeration protein and mediating a conformational change in the agglomeration protein.

Another embodiment of the present invention is directed to a method of evaluating compositions for participation in diseases associated with protein misfolding comprising the steps of: (a) providing a non-human transgenic animal, wherein the transgenic animal expresses an untranslated non-coding RNA capable of binding an agglomeration protein and mediating a conformational change in said agglomeration protein; and (b) placing the composition to be evaluated in said transgenic animal and evaluating the animal for at least one characteristic of disease associated with protein misfolding.

A further embodiment of the present invention is directed to a method of making a non-human transgenic animal expressing characteristics of a disease associated with protein misfolding comprising the steps of: (i) inserting a nucleic acid molecule in an animal cell, wherein the nucleic acid molecule further comprises a promoter joined to a nucleic acid sequence encoding for an RNA capable of binding an agglomeration protein and mediating a conformational change in the agglomeration protein under the control of a promoter, thereby resulting in a non-human transgenic animal cell; and (ii) growing a whole animal from the animal cell, thereby resulting in a non-human transgenic animal expressing the characteristics of a disease associated with protein misfolding.

In particular embodiments, the animal is selected from the group of animals comprising Chordata, Arthropoda, Mullusca and Nematodata. A preferred nematode is Caenorhabditis elegans. Particularly preferred animals selected from the Chordata Phylum is a mouse, rat and certain primates. A particularly preferred animal selected from the Arthropoda phylum is an insect, and, most preferably, Drosophila melanogaster, the common fruit fly.

The untranslated RNA that is expressed is preferably an RNA of twenty to 1,000 nucleotides and most preferably, approximately, 100 to 500 nucleotides. The RNA is preferably complex in the sense of having at least one section forming a hairpin structure and having at least one loop structure. A preferred untranslated RNA has sequences derived from retrovirus. As used herein, the term “untranslated, non-coding” means that the RNA does not code for a complete protein and translated products, proteins, related to such RNA can not be identified in the cell in which such RNA is placed.

For example, without limitation one preferred RNA has at least one section of ten to five hundred nucleotides in a sequence derived from a retrovirus, transposons and transposable elements, or other untranslated portion of the genome. One preferred retroviral sequence is derived from HIV.

Preferably, the over-expressed RNA sequence has at least one section of ten to five hundred nucleotides that are recognized by an RNA dependent RNA polymerase. As used herein, the term “recognized” means that the section allows the RNA to be replicated by the RNA dependent RNA polymerase under conditions in which the enzyme normally replicates RNA templates.

A preferred untranslated expressed RNA corresponds to RQ 11+12. As used herein, the term “corresponds” means having substantially the same nucleotide sequence as, or opposite pairing relationship as in the DNA encoding for such RNA. Preferably, the expressed RNA has twenty nucleotides in a sequence that substantially corresponds to the sequence of RQ 11+12.

Embodiments of the present invention have particular utility for identifying compositions that participate in agglomeration disease processes. A method has the steps of providing an animal that over expresses an untranslated, non-coding RNA. And, the method comprises the step of placing a composition in the animal and evaluating the animal for at least one characteristic of agglomeration disease.

A characteristic of agglomeration disease is preferably selected from the group comprising intra-and extra-cellular aggregates (plaques, filaments, amyloid deposits, fibrils, tangles, and such) formation and behavior abnormalities. Aggregate formation associated with brain disorders is found in association with neurons. Aggregate formation can be found in any region of the brain which is associated with memory, learning, behavior and/or locomotion. Aggregate formation associated with other disorders may be found in arteries or other affected organ systems. Behavior abnormalities include learning and memory impairment. In Drosophila melanogaster systems, behavior abnormalities include courtship behaviors, song or chirping patterns, decreased memory capacity and locomotion impairments. In mammalian systems, including mice, behavior abnormalities are decreased memory, decreased learning capability and locomotion impairments.

Preferably, the animal is selected from the group of animals comprising Chordata, Arthropoda, Mullusca and Nematodata. A preferred nematode is C. elegans. A preferred animal from the Arthropoda phylum is selected from the subphylum Insecta, and, is, most preferably, Drosophila melanogaster.

A preferred animal from the Chordata phylum is a mammal and, a preferred mammal is a mouse, rat or primate.

As used herein, “placing” means causing the composition to be placed in contact with, inserted, or injecting the composition into the animal or causing the composition to be ingested or inhaled into the animal or other means.

The RNA that is over-expressed is preferably an RNA of twenty to 1,000 nucleotides and most preferably, approximately, 100 to 500 nucleotides. The RNA is preferably complex in the sense of having at least one section forming a hairpin structure and having at least one loop structure. A preferred RNA has sequences derived from retrovirus.

For example, without limitation one preferred RNA has at least one section of ten to five hundred nucleotides in a sequence derived from a retrovirus transposons and transposable elements, or other untranslated portion of the genome. One preferred, retroviral sequence is derived from HIV.

Preferably, the expressed and untranslated RNA sequence has at least one section of ten to five hundred nucleotides that are recognized by an RNA dependent RNA polymerase. As used herein, the term “recognized” means that the section allows the RNA to be replicated by the RNA dependent RNA polymerase under conditions in which the enzyme normally replicates RNA templates.

A preferred expressed and untranslated RNA corresponds to RQ 11+12. As used herein, the term “corresponds” means having substantially the same nucleotide sequence as, or complementary pairing relationship as in the DNA encoding for such RNA. Preferably, the over-expressed RNA has twenty nucleotides in a sequence that substantially corresponds to the sequence of RQ 11+12.

Embodiments of the present invention feature a non-naturally occurring animal transformed to over express a non-coding RNA. These and other features and advantages of the present invention will be apparent to those skilled in the art upon viewing the figures and studying the detailed description that follow.

### BRIEF DESCRIPTION OF THE FIGURES

FIG. 1 depicts the construction of an expression vector.

FIG. 2 depicts, in diagram form, an experimental design for the transformed animals compared to a control animal.

FIG. 3 depicts RQ 11+12 RNA.

FIG. 4 shows a schematic representation of the predicted secondary structure of RQ11+12 (FIG. 4A) and RQT157 (FIG. 4B) RNAs. The position of Rev-binding element (RBE) and the sarcin/ricin-cleavage domain (S/R) are indicated in RQ11+12 RNA.

FIG. 5A shows an electrophoretic mobility-shift assay for RQ11+12 RNA reacted with protein extracts from fly thoraxes (lane 1); rat liver (lane 2); fly heads (lane 3); and rat brain (lane 4). The diffuse labeling observed in the lanes 3 and 4 probably resembles binding of RQ11+12 RNA with various proteins differing by molecular mass.

FIG. 5B shows a Northern analysis of heat-shock induced RNA expression in transgenic flies. Lane 1: RQ2 homozygous flies raised under normal temperature condition (control). Lane 2: RQT heterozygous flies raised under normal temperature condition (control). Lane 3: RQ2 flies immediately after 30 min of heat shock at 37° C. Lane 4: RQT flies immediately after 30 min of heat shock at 37° C. Lane 5: RQ2 flies after 5 hours of recovery (after 30 min. heat shock at 37° C.). Lane 6: RQT flies after 5 hours of recovery (after 30 min. heat shock at 37° C.). Lane 7: RQ2 flies after 15 hours of recovery (after 30 min. heat shock at 37° C.). Lane 8: RQT flies after 15 hours of recovery (after 30 min. heat shock at 37° C.).

FIG. 6 shows Congo Red-positive aggregates and neuropathology. Congo Red stained salivary glands of non-transgenic (panel a; normal) and RQ2 transgenic (panel b; aggregates are indicated by an arrow). Stained central complex of non-transgenic (panel c; normal) and RQ2 transgenic (panel d; aggregates are indicated by an arrow). Congo Red stained optic lobes of non-transgenic (panel e; normal) and Geimsa stained RQ2 transgenic (panel f; needle-like and filamentous structures are indicated by arrows). Panel g shows dying nerve cells (A and B as indicated by arrows), in the frontal protocerebral cellular mass in the brain of RQ2 transgenic strain. The Giemsa positive, needle-like structures are seen nearby the dying cells. Magnification: 10×100, immersion. Panel h shows pycnomorphic cells in the frontal protocerebral cellular mass in the brain of RQ2 transgenic flies with the Giemsa-positive filamentous inclusions. Magnification: 10×100, immersion.

FIG. 7 is a set of graphs representing learning indices (LI) in wild type and transgene strains. Abscissa: time after training (minutes). Ordinate: LI—learning index. Sample size for each time point —20 males. The (*) represents LI after heat shock which significantly differs from LI at 25° C. (one-sided randomization test, α_{R}<0,05). The (&) represents LI which is significantly lower than that of Canton-S strain under similar conditions (one-sided randomization test, α_{R}<0,05). The (@) represents LI which is significantly lower than that of RQT157 strain under similar conditions (one-sided randomization test, α_{R}<0,05). The (#) represents LI at 3 hrs after training which significantly differs from LI 0 hrs after training (one-sided randomization test, α_{R}<0,05). The (ˆ) represents LI after HS1 or HS2 which significantly differs from LI after HS (one-sided randomization test, α_{R}<0,05).

### DETAILED DESCRIPTION OF THE INVENTION

The present invention provides model systems that exhibit cellular and/or physiological characteristics of protein misfolding diseases. The model systems include cells or whole organisms expressing RNA molecules that mediate protein misfolding. In the model systems of the present invention, the expressed RNA induces normal proteins to misfold into agglomerated proteins that mimic the misfolded proteins found in naturally-occurring cells or organisms having agglomeration diseases. The model systems of the present invention are useful for screening and/or identifying agents that modulate (e.g., increase or decrease) the protein agglomeration process. The present invention also provides methods for producing the model systems.

In certain embodiments, the present invention is directed to a non-human transgenic animal for use in the study of diseases associated with protein misfolding, wherein the transgenic animal expresses an untranslated non-coding RNA capable of binding an agglomeration protein and mediating a conformational change in the agglomeration protein.

Another embodiment of the present invention is directed to a method of evaluating compositions for participation in diseases associated with protein misfolding comprising the steps of: (a) providing a non-human transgenic animal, wherein the transgenic animal expresses an untranslated non-coding RNA capable of binding an agglomeration protein and mediating a conformational change in said agglomeration protein; and (b) placing the composition to be evaluated in said transgenic animal and evaluating the animal for at least one characteristic of disease associated with protein misfolding.

A further embodiment of the present invention is directed to a method of making a non-human transgenic animal expressing characteristics of a disease associated with protein misfolding comprising the steps of: (i) inserting a nucleic acid molecule in an animal cell, wherein the nucleic acid molecule further comprises a promoter joined to a nucleic acid sequence encoding for an RNA capable of binding an agglomeration protein and mediating a conformational change in the agglomeration protein under the control of a promoter, thereby resulting in a non-human transgenic animal cell; and (ii) growing a whole animal from the animal cell, thereby resulting in a non-human transgenic animal expressing the characteristics of a disease associated with protein misfolding.

The model cell or organism of the present invention carries at least one copy of a nucleic acid molecule (transgene) encoding the RNA molecule that mediates protein misfolding. The cellular machinery of the model cell or organism transcribes the transgene into the RNA molecule so as to express the RNA molecule.

In certain embodiment, the disease associated with protein misfolding is Alzheimer's disease.

In particular embodiments of the present invention, the agglomeration protein is an amyloid protein. In more preferred embodiments, the amyloid protein is a tau protein or a 3-amyloid protein.

The model cell can be from any animal including, but not limited to, Chordata, Arthropoda, Nematodata and Mullusca. The model organism can be any animal including, but not limited to, Chordata, Arthropoda, Nematodata and Mullusca.

Embodiments of the present invention will be described in detail with respect to Drosophila melanogaster and mouse systems with the understanding that other animal and insect systems can be readily employed as well.

One embodiment of the present invention is directed to a model system for agglomeration disease comprising an animal or an insect that expresses an untranslated, non-coding RNA. The animal or insect expressing the RNA can be male or female. Animals or insects are normally selected for their similarity to humans and/or the ease of transforming such animal or insect, and the speed in which the animal or insect matures. Common animal or insect systems are selected from the group of animals comprising Chordata, Arthropoda, Nematodata and Mullusca. A particularly preferred animal selected from the Chordata Phylum is a mouse, rat and several species of primates. A particularly preferred animal selected from the Nematodata Phylum is C. elegans. A particularly preferred animal selected from the Arthropoda phylum is an insect, and, most preferably, Drosophila melanogaster, the common fruit fly. The common fruit fly is particularly useful because it matures rapidly and is relatively easy to transform, control and maintain.

The untranslated RNA that is expressed is preferably an RNA of twenty to 1,000 nucleotides and most preferably, approximately, 100 to 500 nucleotides. The RNA is preferably complex in the sense of having at least one section forming a hairpin structure and having at least one loop structure. As used herein, the term “hairpin” refers to a structure of RNA in which the RNA molecule folds onto itself and exhibits affinity for complementary sequences within the molecule. A “loop” is a section of the RNA molecule between to sections which exhibit affinity for each other. That is, the loop is a section of the RNA between two sections form a single hairpin structure.

These complex structures are found in naturally occurring RNA of viral origin. A preferred RNA has sequences derived from retrovirus. However, it not necessary for the entire retroviral sequence to be used. As used herein, the term “non-coding” means that the RNA does not code for a complete protein and translation products related to such RNA can not be identified in the cell in which such RNA is placed.

One preferred RNA has at least one section of ten to five hundred nucleotides in a sequence derived from a retrovirus. One preferred, retroviral sequence is derived from HIV.

The RNA molecule induces a protein to misfold. The protein can be a normal or mutant protein. In one embodiment, the misfolded protein is a protein-RNA complex which includes the misfolding-inducing RNA. The protein can be any protein associated with a protein misfolding disease including, but not limited to, neurodegenerative, Alzheimer's, Huntington's, Parkinson's, polyglutamine-associated, tau-associated, scrapie, mad cow, chronic wasting, and spongiform encephalopathies. The protein misfolding disease can affect any organ including brain, lung, heart, kidney, pancreas, or arteries. The protein can be a prion or tau protein.

Preferably, the expressed untranslated RNA sequence has at least one section of ten to five hundred nucleotides that are recognized by an RNA dependent RNA polymerase. As used herein, the term “recognized” means that the section allows the RNA to be replicated by the RNA dependent RNA polymerase under conditions in which the enzyme normally replicates RNA templates.

A preferred expressed untranslated RNA corresponds to RQ 11+12. As used herein, the term “corresponds” means having substantially the same nucleotide sequence as, or complementary pairing relationship as in the DNA encoding for such RNA. Preferably, the over-expressed RNA has twenty nucleotides in a sequence that substantially corresponds to the sequence of RQ 11+12. The RNA RQ 11+12 is a Q-beta replicase template containing HIV-Rev binding element (underlined in the sequence presented below), and the Sarcin/Ricin cleavage domain (italicized in the sequence presented below). The sequence for the RNA RQ 11+12 is set forth in Sequence No 1.

Sequence No. 1

5′GGGGUUUCCAACCGGAAUUUGAGGGAUGCCUAGGCAUCCCCCGUGCGU
CCCUUUACGAGGGAUUGUCGACUCUAGUCGACGUCUGGGCGAAAAAUGUA
CGAGAGGACCUUUUCGGUACAGACGGUACCUGAGGGAUGCCUAGGCAUCC
CCGCGCGCCGGUUUCGGACCUCCAGUGCGUGUUACCGCACUGUCGACCC-
3′

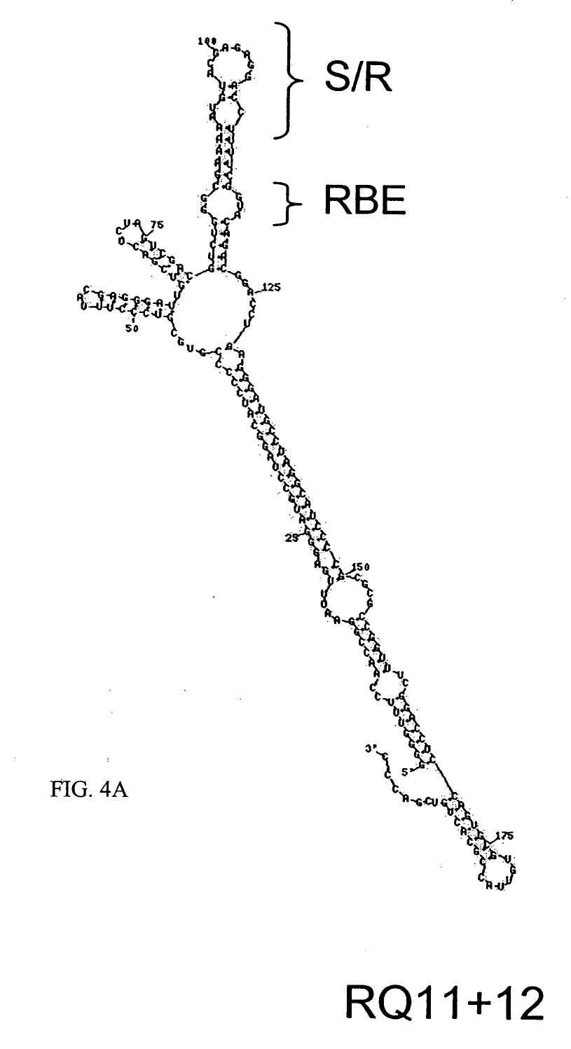

This sequence is also depicted in FIG. 3.

In another embodiment, the RNA can be RQT 157 which differs from RQ11+12 by a 40 nucleotide deletion (Zeiler, et al., 2003 Biotechnol Appl Biochem 37:173-182). The RQT157 RNA lacks the sarcin/ricin cleavage domain and the HIV-Rev binding element.

The RNA, which mediates protein misfolding, is replicable with a nucleic acid polymerase such as a DNA polymerase or RNA polymerase. The RNA polymerase can be a Q-beta replicase from bacteriophage Q-beta (Haruna and Spiegelman 1965 Science 150:884-886) or brome mosaic virus replicase (March et al., 1987 in “Positive Strand RNA Viruses” Alan R. Liss, N.Y.), or any derivative or modified replicase thereof. Q-beta replicase comprises eukaryotic elongation factor Ts (Ef−Ts), eukaryotic elongation factor Tu (Ef−Tu), S1 nuclease, and a replicase component. The RNA polymerase can be a modified form of Q-beta replicase, such as Q-Amp™ (Q-RNA, Inc., New York, N.Y.).

Embodiments of the present invention have particular utility for identifying compositions that participate in protein agglomeration disease processes. A method has the steps of providing an animal that over expresses an untranslated, non-coding RNA. And, the method comprises the step of placing a composition in said animal and evaluating the animal for at least one characteristic of agglomeration disease.

A characteristic of agglomeration disease is preferably selected from the group comprising aggregate formation and behavior abnormalities. Aggregate formation associated with brain disorders is found in association with neurons. Aggregate formation associated with other disorders may be found in arteries or other affected organ systems. Behavior abnormalities include learning and memory impairment. In Drosophila melanogaster systems, behavior abnormalities include courtship behaviors, song or chirping patterns, decreased memory capacity and locomotion impairments. In mammalian systems, including mice, behavior abnormalities are decreased memory, decreased learning capability and locomotion impairments.

In one embodiment the animal is selected from the group of animals comprising Chordata, Arthropoda, Nematodata and Mullusca. A preferred animal from the Arthropoda phylum is selected from the subphylum Insecta, and, is, most preferably, Drosophila melanogaster.

A preferred animal from the Chordata phylum is a mammal and, a preferred mammal is a mouse, rat or primate.

As used herein, “placing” means causing the composition (i.e., nucleic acid molecule encoding the RNA) to be placed in contact, inserted or ingested the composition into the cell or animal or causing the composition to be injected or inhaled into the animal or other means. Injection of a composition can be done intramuscularly, intravenously, into the body cavity or otherwise.

The copy of the nucleic acid molecule (transgene) can be present in the somatic or germline cells of the whole organism. The transgene, which encodes the RNA molecule, can be stably integrated into the genome of the model cell or organism, or can be exist in a transient (non-integrated) state. The RNA can be expressed in any type of cell. The RNA molecule can be expressed in any tissue, organ or body part of the organism.

The recipient cell or organism into which the composition is placed can be any wild type or mutant strain. For producing transformed Drosophila, the recipient cell (e.g., egg or embryo) can be a Canton-S or y w[67c23] or any strain that permits P-element transposition.

The RNA that is expressed is preferably an RNA of twenty to 1,000 nucleotides and most preferably, approximately, 100 to 500 nucleotides. The RNA is preferably complex in the sense of having at least one section forming a hairpin structure and having at least one loop structure. A preferred RNA has sequences derived from retrovirus. As used herein, the term “non-coding” means that the RNA does not code for a complete protein and transcription products related to such RNA can not be identified in the cell in which such RNA is placed.

For example, without limitation one preferred RNA has at least one section of ten to five hundred nucleotides in a sequence derived from a retrovirus transposable element or transposon or other untranslated portion of the genome. One preferred, retroviral sequence is derived from HIV.

Preferably, the expressed untranslated RNA sequence has at least one section of ten to five hundred nucleotides that are recognized by an RNA dependent RNA polymerase. As used herein, the term “recognized” means that the section allows the RNA to be replicated by the RNA dependent RNA polymerase under conditions in which the enzyme normally replicates RNA templates.

A preferred expressed untranslated RNA corresponds to RQ 11+12. As used herein, the term “corresponds” means having substantially the same nucleotide sequence as, or opposite pairing relationship as in the DNA encoding for such RNA. Preferably, the expressed RNA has twenty nucleotides in a sequence that substantially corresponds to the sequence of RQ 11+12.

Embodiments of the present invention feature a non-naturally occurring animal or insect cell transformed to express a non-coding RNA. One further embodiment comprises and method of making a transformed whole animal or insect that expresses a non-coding RNA the method comprises the steps of placing a nucleic acid molecule encoding the RNA under the control of a promoter in an animal or insect cell, and growing a whole animal or insect from the cell. The method further comprises the step of placing the animal, insect or cell having the non-coding RNA under conditions in which the RNA is transcribed.

The nucleic acid molecule encoding the RNA can be placed in the cell (e.g., egg or embryo) as part of a vector. Vectors, include plasmids, cosmids and phagemids. The vector can be autonomously replicating in prokaryote or eukaryote cells. The vector can be an expression vector, comprising various expression control sequences such as: a promoter sequence, secretion signal, enhancer, transcription initiator, transcription terminator, poly-A sequence, translation initiator, translation terminator, and other transcriptional or translational regulatory elements. The vector can include one or more poly-linker sequences. The vector can be based on well known vectors, such as pUC, Casper, or Bluescript.

The vectors can include sequences encoding a selectable marker, such as eye or body color, or produces abnormal phenotypes such as changes in appendages, wings or bristles. In one embodiment, the marker can be a gene sequence for eye color, such as y w[67c23]. The selectable markers can encode drug resistance, enzyme, fluorescent compound, a bioluminescent compound, chemiluminescent compound, a chromophore, a metal chelator, or biotin.

The promoters can be inducible, conditional, or tissue-specific promoter sequences, including those from the genes for heat shock proteins, eye color, body color, isopropyl-β-D-thiogalactopyranoside (e.g., IPTG), GAL4-UAS, alcohol dehydrogenase, isocytochrome C, acid phosphatase, enzymes associated with nitrogen catabolism, and enzymes responsible for maltose and galactose utilization. The promoter can be a baculovirus polyhedrin promoter for expression in insect cells.

The vector can include any P-element sequence for transposition of the transgene into the genome of the recipient cell or egg. The P-element can include a gene sequence encoding a transposase activity (in cis). Alternatively, the transposase activity can be provided by a separate vector, such as a helper vector (in trans). In one embodiment, the helper vector is Turbo delta 2-3.

Methods for generating a recombinant expression vector encoding the modified hepsin molecules of the invention are well known in the art (T Maniatis, et al., 1989 Molecular Cloning, A Laboratory Manual, Cold Spring Harbor Laboratory, Cold Spring Harbor, N.Y.; F Ausubel, et al. 1989 Current Protocols in Molecular Biology, John Wiley & Sons, New York N.Y.). A preferred inducible promoter for use with lower forms of animals such as Drosophila melanogaster is a heat shock promoter. In one embodiment, the heat shock promoter is an hs70 promoter. A preferred inducible promoter for use with higher animals, such as mouse systems comprise of antibiotics or heat shock inducible promoter.

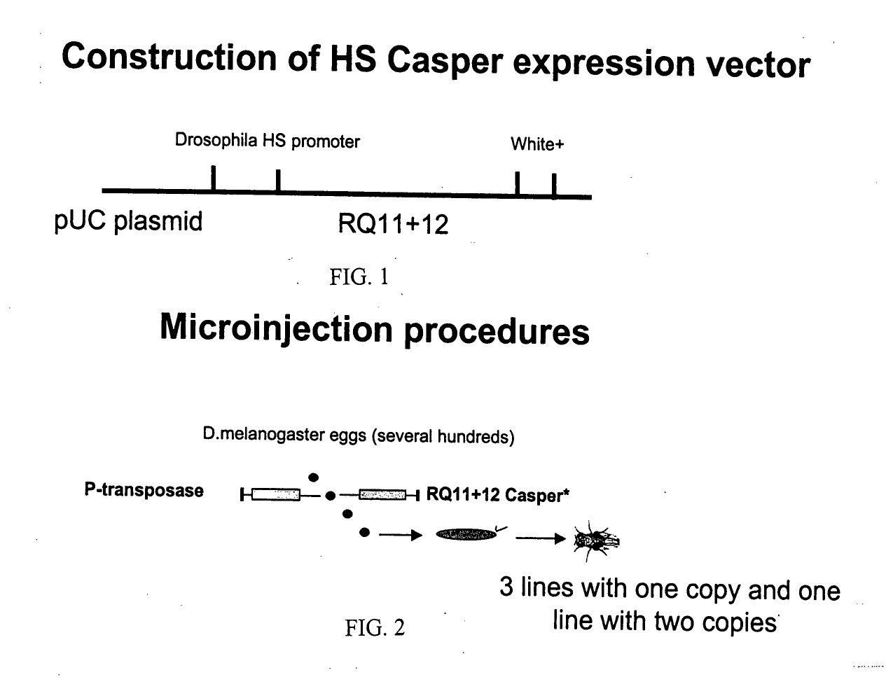

The non-coding RNA and promoter are preferably incorporated in a vector. A preferred vector for placing such RNA in Drosophila melanogaster is a Casper expression vector. The Casper expression vector is based on the well-characterized pUC vector. The construction of this vector is depicted in FIG. 1.

The vector can be placed in a cell, egg or embryo using any method well known in the art, including microinjection, electroporation, calcium phosphate mediated precipitation, liposome fusion, retroviral infection, particle-gun bombardment or other means. Drosophila carrying the transgene can be produced using P-element mediated germ line transformation methods (Rubin et at., 1982 Science 218:348-353) or other methods (O'Brochta et al., 1996 Insect Biochem. Mol. Biol. 26:739-753; or Louleris et al., 1995 Science 270:2002-2005). The transgene can be inherited in a normal Mendelian fashion

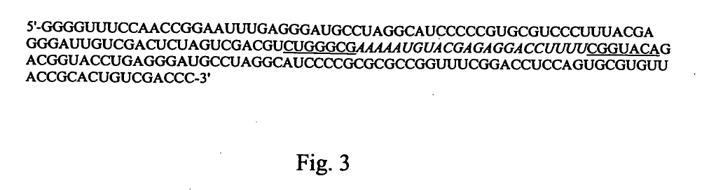

The vector is preferably placed in the cell by microinjection techniques. The process is depicted in FIG. 2. Normally, many cells are transformed by microinjection and those that successfully receive the vector replicate and develop further. For example, without limitation, Drosophila melanogaster cells which receive the vector will develop into larva, pre-pupae and finally adult flies.

The transformed cell or whole organism carrying the transgene can be induced to express the RNA using any inducing condition that stimulates the inducible promoter, including heat shock, IPTG, or sugar (e.g., galactose or maltose). For example, the heat shock condition includes a range of temperatures from 25 to 40 degrees C. The inducing condition can be applied to the model cell or whole organism at any developmental time period. In one embodiment, the transformed Drosophila can be induced during any stage including embryo, larvae, eclosion, pupae, or adult.

The expression of the non-coding RNA is controlled by the temperature at which the transformed Drosophila melanogaster is maintained. Subjecting the transformed insect to a temperature of approximately 37 degrees centigrade causes the expression of the non-coding RNA.

The transformed animal is preferably allowed to mature. Preferably, the animal is evaluated with respect to suitable controls to study the effects of the non-coding RNA. The expression of non-coding RNA causes pathologies and behavior changes that are of the same nature and kind as exhibited by animals with agglomeration disease.

The transformed organism can be selected based on certain phenotypic traits, including color, shape or appearance of eye, body, appendages, wings or bristles. The transformed organism can be backcrossed to produce future generations carrying more than one copy of the transgene, or to rid the suppressive effects of certain mutations. The transformed organism can carry at least one copy (heterozygous) or two copies (homozygous) of the transgene.

The transformed cell or organism can be analyzed for the presence of the transgene using any DNA detection method, including: Southern blot, DNA dot blot, PCR, or DNA sequencing. The RNA which is expressed in the model cell or organism can be detected using any RNA detection assays, including: Northern blot, RNA gel shift, RNA dot blot, S1-nuclease, whole animal in situ, tissue in situ, and the like. The presence of the RNA can also be indicated by the presence of agglomerated protein in a cell, tissue or organ. Additionally, the presence of misfolded proteins in the cell, tissue or organ, can be detected by performing known procedures that reveal plaques, filaments, amyloid deposits, fibrils, or tangles. The tissue or organ can be analyzed for the presence of dying cells which may correlate with the presence of misfolded proteins. For example, the cell, tissue or organ can be reacted with any histological stain such as Congo red, thioflavin, Giesma, or hematoxylin. The misfolded protein can occur intracellularly or extracellularly.

Any part of the transformed organism at any developmental stage can be analyzed for the presence of the transgene (e.g., DNA encoding the RNA) or the expressed RNA. The parts of the transformed animal to be analyzed include: salivary glands, imaginal discs, fat body, mucosa, gut, muscles, brain, central complex, frontal protocerebral cellular mass, optical lobes, and nerve cells.

The transformed animal can be used to evaluate potential treatments and drug candidates. The ability of a composition to alter the progression of pathology and behavior changes over time may suggest a potential drug candidate.

These and other features and advantages will be described now with respect to the following Examples.

### EXAMPLES

The following examples are presented to illustrate the present invention and to assist one of ordinary skill in making and using the same. The methodology and results may vary depending on the intended goal of treatment and the procedures employed. The examples are not intended in any way to otherwise limit the scope of the invention.

### Example 1

### Construction of a Vector for Drosophila Melanogaster

The pUC plasmid was modified to incorporate a DNA construct comprising the Drosophila heat shock promoter, the DNA for RQ 11+12, linked to a gene for white eyes in a manner known in the art. An expression vector, HS Casper, with these features is depicted in FIG. 1. This vector is multiplied in E. coli and harvested in the manner known to individuals skillful in art.

The following provides a description of the methods used to produce transgenic Drosophila carrying and expressing RNA molecules that mediate protein folding. In the transgenic Drosophila, the misfolded proteins cause symptoms known to be associated with protein agglomeration diseases.

Material and Methods

Experimental Animals

Stocks of Drosophila melanogaster were maintained at 25 degrees C. on a standard yeast-raisin medium in a 12 hour light/12 hour dark cycle. Flies of four strains were used in these experiments: wild type Canton-S(CS), y w67c23 (J Rubin and A Spradling 1982 Science 218: 348-353) strain used to develop transgenic strains, and two transgenic strains, i.e., a transgenic strain with two transgene RQ11+12 RNA inserts (RQ2-flies) and a transgenic strain with two transgene RQT157 RNA inserts (RQT-flies). Canton-S served as a control strain to study the effect of an shsRNA (small, highly structured RNA), RQ11+12 or RQT157, on fly behavior and aggregate formation. RQT flies served for identification of the particular active portion within RQ11+12 RNA.

Vector Constructs and RNA Compositions

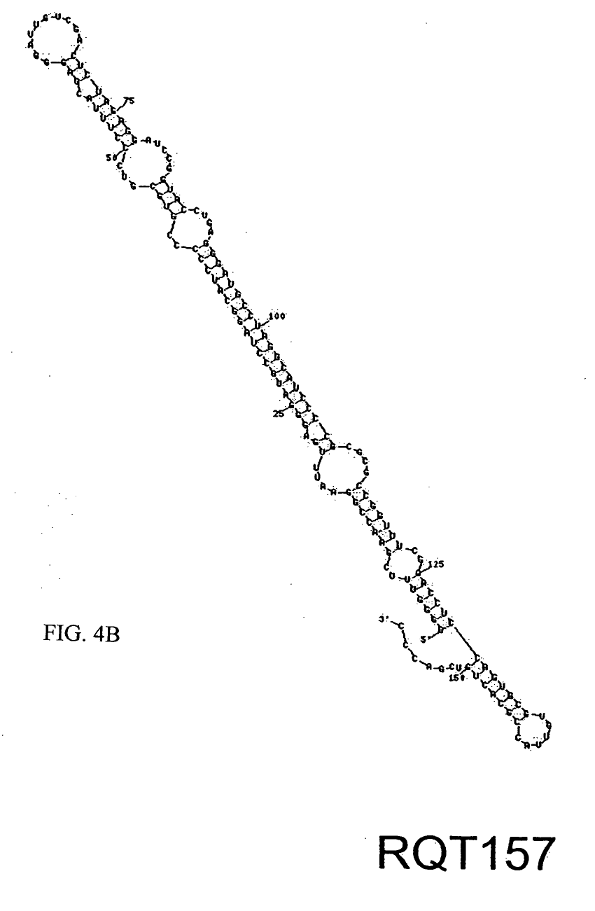

Two shsRNAs, RQT157 and RQ11+12, were used for construction of transgenic flies. These RNAs were selected from the RNA collection described previously in detail (B Zeiler, et al., 2003 Biotechnol Appl Biochem 37:173-182; V Adler, et al., 2003 Mol Biol, 332: 47-53). The sequence of RQ11+12 RNA differs from that of RQT157 by the Rev Responsive Element from HIV-1 (S Iwai, et al., 1992 Nucl Acid Res 20: 6465-6472) and the Sarcin/Ricin cleavage domain (Y Endo and I G Wool 1982 J Biol Chem 257: 9054-9060). The sequences encoding RQ11+12 and RQT157 RNAs were amplified by PCR using primers TTTGAATTCAGGGGTTTCCAACCGGA and TAATCTAGAGGGTCGACAGTGCGGTAA, and plasmids PUC-RQ11+12 and pUC-RQT157 as templates, respectively. The resulting PCR fragments were cloned into EcoRI and XbaI restriction sites of pCaSpeR-hs vector described previously (V Pirotta 1988 in: “Vectors: A Survey of Molecular Cloning Vectors and Their Uses”, eds. R Rodrigues and D Denhardt, Butterworth, Boston, pp 437-445). Both RNAs contain 218 bases of hsp 70 5′ UTR (untranslated region) and 401 bases of hsp 70 3′ UTR (untranslated region) and polyadenylation signal site. Nucleotide composition and the thermodynamically most favorable secondary structures of RQ11+12 (RQ) and RQT157 (RQT) shsRNAs created with RNA draw (O Matzura and A Wennborg 1996 Computer Applications in the Biosciences 12: 247-249) are presented in the FIG. 4.

P-Element-Mediated Transformation

Plasmid DNA for transformation of D. melanogaster was purified by HiSpeed™ Plasmid Midi Kit (Qiagen) and used for embryo injection as described (J Rubin and A Spradling 1982 Science 218: 348-353). Transposase activity was provided by the helper plasmid Turbo D2-3 (H M Robertson, et al., 1988 Genetics 118: 461-470), and the recipient embryos were from the D. melanogaster y w67c23 strain. Adults emerging from the injected embryos were crossed with y w67c23 flies of the opposite sex, and the eye color of their progeny was examined. Transformed lines that were homozygous for the transgene were established by full-sib mating. The presence of homozygous transgene copies was confirmed by Southern blotting and in situ hybridization as described (J K Lim 1993 Dros Inf Serv 72:73-77). To get rid of the known suppressive effect on behavioral displays of the yellow mutation, y was out crossed from the strains to be tested.

Heat Shock Treatment

Previously designed heat shock (HS) treatment protocol (E A Nikitina, et al., 2003 Russian Journal of Genetics 39: 25-31) was used either to modulate a HS stress response in adult flies, or to induce an expression of a HS-driven gene at certain developmental stages, crucial for the formation and function of the brain structures implicated in learning acquisition and memory retention of the adult fly. According to this protocol, behavioral patterns, brain morphology and/or immunocytochemistry of adult 5 day old flies were assessed after the HS treatment was applied either to larvae on an embryo stage during formation of the mushroom bodies (HS1 group,), or to larvae III-pupa during formation of the central complex of the brain (HS2 group), or to adult flies, one hour before a test (HS group).

Adult males were collected without anesthesia for behavioral or histological experiments and kept individually till the age of 5 days. Prior experiments, the flies were transferred into empty, pre-heated vials for 30 minutes at 37 degrees C. in a water bath. Then they were kept at 25 degrees C. for 1 hour before the experiment started. For studies of remote effects of HS treatment and Q-RNA expression during Drosophila development, adult flies were allowed to lay eggs for 5 hours, the parents were then discarded and first instar larvae or prepupae were subjected to heat shock as described above. After eclosion, newly hatched males were collected and staged as above till the experiment at the age of 5 days.

RNA Preparation and Analysis

RNA from control and transgenic larvae and flies was prepared by standard method with isothiocyanate (P Chomczynski and N Sacchi 1987 Anal Biochem 162: 156-159), separated by agarose and transferred to a membrane for hybridization (J Sambrook, et al., 1989 in: “Molecular Cloning: A Laboratory Manual” Cold Spring Harbor Laboratory Press, Cold Spring Harbor, N.Y.) with a ³²P-labeled PCR fragment containing the appropriate insert. Hybridization was carried out overnight at 42 degrees C. in 50% formamide, followed by two 20 minutes washes in 2×SSC, 0.2% SDS at 42 degrees C., two 20 minute washes in 1×SSC, 0.2% SDS at 42 degrees C., and one 20 minutes wash in 0.2×SSC, 0.2% SDS at 68 degrees C. Labeling of RQ11+12 RNA and gel shift analysis were performed as described (D Garbuz et al., 2003 J Exp Biol 206: 2399-2408).

Histochemical Analysis

In histopathology studies Canton-S, RQ2 and RQT adults were placed in mass-histology “collars” as described (M Heisenber and K Böhl 1979 Z. Naturforsch. 34c: 143-147), fixed for 3 hours in 4% paraformaldehyde embedded in paraffin and sliced into 7 micro meter serial frontal sections. Each collar contained heads of HS-treated (HS1, HS2, and HS groups) and non-treated adult flies providing simultaneous and uniform fixation and histological treatment of the specimens. The sections were de-paraffinized in three changes of xylene, 5 minutes each, dehydrated through graded alcohols and stained according to Putchler's modification of Congo red stain for amyloid aggregates

Larval tissues, including salivary glands and brains, were dissected in solution of 0.1% Triton X-100 in PBS pH 7.5, fixed in solution of 3.7% paraformaldehyde and 1% Triton X-100 in PBS pH 7.5. Lightly squashed preparations were stained with Mayer's Hematoxylin for 5 minutes, washed in water until the appearance of distinct blue staining. The slides were placed in working NaCl solution, microwaved for 45 seconds at 20 power and placed directly into working Congo red solution, microwaved, 20 power, for 45 seconds. The slides were allowed to sit in this solution for 3 minutes, dehydrated rapidly in 3 changes of absolute alcohol, cleared in xylene and mounted with the cover slips using Entellan-New media. On the slides, nuclei were seen as blue mass due to Mayer's hematoxylin staining, whereas amyloid aggregates were distinctly stained as pink to red inclusions.

Observing Courtship Behavior

Experimental flies were collected without any anesthesia soon after eclosion. Males were kept individually in culture vials with yeast-raisin medium for 5 days at 25 degrees C. until the experiment was performed, and females—in small groups (10-15 per vial). For observation of courtship, a male of a strain under study and a virgin 5-days-old wild type female were introduced by shaking through a funnel into different halves of a Perspex experimental chamber (15 mm in diameter, 5 mm high) separated by sliding opaque partition. After several minutes given for adaptation the partition was withdrawn, and the flies were left together. The observation time was 10 minutes. The following parameters were registered: the latencies of courtship and copulation; courtship duration equal to the difference of latter parameters, the percentage of pairs copulated within 10 minutes of observation which served as a criterion of courtship success or efficiency. Usually 20 pairs of flies were tested in each strain. All the experiments were performed at 25 degrees C.

Recording Courtship Sounds

To record sounds emitted by males during courtship of a female, a pair of 5-days-old flies—a male of a given strain and a female—were introduced in the same way as described above into a smaller experimental chamber (8 mm in diameter, 4 mm high) of similar design but with the floor made out of a silicon net with meshes 0.6 mm wide. The experimental chamber with flies was then placed above the membrane of the specially made velocity sensitive microphone. Four such microphones were mounted in the same box for simultaneous recording of singing of 4 pairs of flies. The microphones and the amplifiers were made by an engineer, K. Öchsner in the workshop of Biozentrum of Würzburg University. The scheme, proposed by Bennet-Clark, (H C. Bennet-Clark, “A particle velocity microphone for the song of small insects and other acoustic measurements, J. Exp Biol 108:459-463(1984)), served as a basis and was modified according to our demands to improve the signal-to-noise ratio and to increase the amplification and deepness of filtration (N G Kamyshev, et al., 1999 Learning and Memory 6: 1-20). Due to very effective filters, only sounds in the frequency band 100-800 Hz were recorded. The microphones were mounted inside a foam plastic box (25×25×30 cm, walls 3.7 cm thick) situated in a sound-proof room. A glass window in the cover of the box allowed observation of the behavior of flies during sound recording. The head of the electronic thermometer (Greisinger electronic GTH 175/MO) was mounted at the level of the microphones. All recordings were made at 25±0.5 degrees C. for 5 minutes. The output signals from 4 amplifiers were fed to multi-channel analogue to digital converter of a PC computer and were recorded as sound files (11025 Hz, 8 bit) with the help of the computer program (N G Kamyshev, et al., 1999 Learning and Memory 6: 1-20). In the study of each strain, the courtship object was a virgin Canton-S female.

Analysis of Courtship Sounds

The sound files were analyzed with the help of computer program created by N. G. Kamyshev and P. V. Ozerskii. It allowed the separation of the pulse song and the sine song from extraneous noise (i.e., from sounds which accompany walking, jumps, preening, etc.), to measure the song parameters and to store them in separate Excel files. To evaluate courtship intensity we calculated the singing index (the percentage of time spent in singing over an observation period of 5 minutes).

Testing Learning and Memory

Experimental males were collected without any anesthesia 0-10 hours after eclosion and kept separately in small food vials until the tests were carried out on the fifth day of adult life. For training a naive male (one having no experience of sexual contacts) was placed into the experimental chamber described above together with a 5-day-old mated Canton-S female for 30 minutes. In the test, a trained male was placed into a fresh chamber together with another mobile, mated, female. Naive males were used as a control. The tests were performed at 25 degrees C. either immediately (to test for learning acquisition), or 3 hours after training (to test for memory retention). The time spent in courtship (orientation, following, wing vibration, licking and attempted copulation) was recorded for 300 seconds by pressing an appropriately coded key on a computer, using a specially designed program. J Sambrook, et al., “Molecular Cloning: A Laboratory Manual” Cold Spring Harbor Laboratory Press, Cold Spring Harbor, N.Y. (1989). The resulting courtship index, CI, (percentage of time spent in courtship) was calculated for each male. The learning index (LI) was computed according to the formula of Gailey et al. (D A Gailey, et al., 1984 Genetics 106: 613-623):
9LI=[(CIₙₐ−CIₜᵣ)/CIₙₐ]×100=(1−CIₜᵣ/CIₙₐ)×100
where CIₙₐ and CIₜᵣ are the mean courtship indices for independent samples of naive and trained males, respectively. Statistical comparisons of behavioral data were made by a one-sided randomization test, by directly computing the probability of rejection of the null-hypothesis α_{R}. The sampled randomization test with 10,000 permutations was used. The null-hypothesis was rejected at α_{R}<0.05.
Results
RNA Gel-Shift Assays

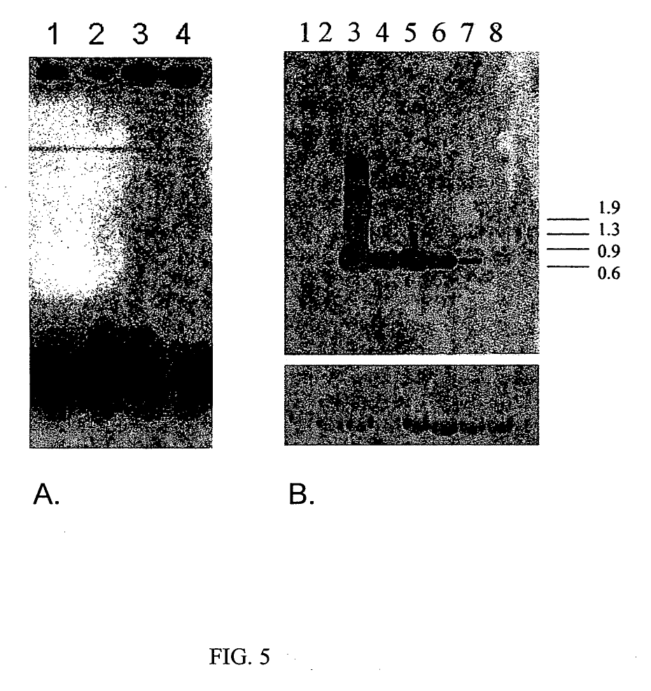

To demonstrate that shsRNA RQ11+12 can interact with Drosophila proteins as it does with human, mouse, rat and hamster cellular prion protein (PrP^{C}), we performed RNA gel-shift assays using the brain homogenates derived from D. melanogaster adult flies (FIG. 5A). These results confirmed that RQ11+12 has affinity for the Drosophila brain proteins (lane 3) and, therefore, potentially can facilitate aggregation of amyloidogenic proteins as was previously demonstrated for prion protein (A Grossman, et al., 2003 Neurochemical Research 28: 955-963).

Northern Analysis

To characterize histological and behavioral effects of shsRNAs in a living organism we generated several transgenic Drosophila strains with RQ11+12 or RQT157 cDNA under D. melanogaster hsp70 promoter. Among the obtained strains most carried a single insertion of RQ11+12. However, one transgenic strain carried a double insertion of RQ11+12 (referred to as “RQ2” strain) which mapped to 57A and 87B region of D. melanogaster polytene chromosomes and a different transgenic strain carried a double insertion of RQT157 at 86F and 3D regions (referred to as “RQT” strain). Preliminary experiments have demonstrated that, probably due to dose effect, both strains with double insertions express more shsRNAs after heat shock. Therefore, we selected the strains having double insertions for use in our experiments. To visualize transgene expression and monitor the steady state preservation of these foreign RNAs in the cells of D. melanogaster, RQ11+12 RNA and RQT157 RNA synthesis was investigated by Northern blot technique at room temperature and at different time-points (30 minutes, 5 hours and 15 hours) after exposure to heat shock (FIG. 5A). Northern blot analysis using labeled RQ11+12 DNA probe after long exposure time detected a residual signal in the transgenic strains kept at room temperature, which was indicative for the slight “leakage” of the heat shock promoter in these strains. As expected, no RQ11+12 expression was detected in Canton-S strain after heat shock treatment and at room temperature. We have also used Northern blot hybridization to demonstrate that RNA in both constructs is expressed after a heat shock treatment at all developmental stages used in the investigation. Abundant RQ11+12 and RQT157 RNA expression was evident after temperature elevation both in adult males and females (FIG. 5B) as well as at larval stages I and III used in the investigation. This expression remained rather prominent 5 hours of recovery following heat shock treatment (lanes 5 and 6) and was still present (though becoming weaker) even after 15 hours of recovery (lanes 7 and 8).

Histological Evidence of Amyloid Aggregates and Neural Pathology

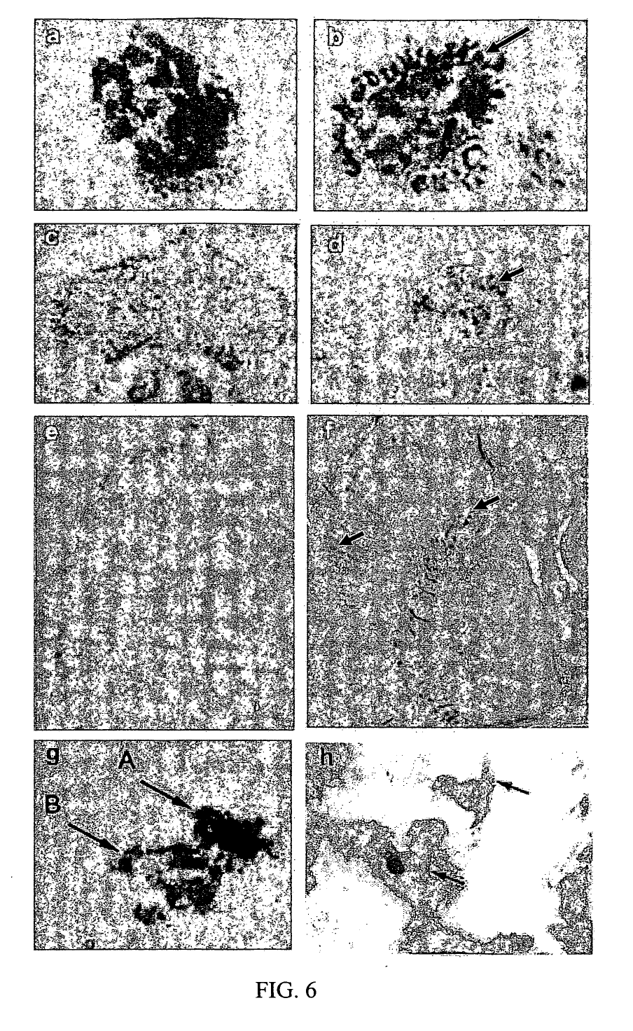

The hallmark of protein misfolded neurodegenerative disorders such as Alzheimer's, Parkinson's, and Huntington's diseases, the polyQ and prion diseases, is the formation of aggregates which typically include misfolded, partially insoluble, amyloidogenic proteins enriched with beta-sheet structures. These aggregates characteristically stain with amyloid-specific histological stains, such as thioflavines and Congo red (C M Dobson 2003 Nature 426: 884-890). We used Congo red (CR) staining for the visualization of putative shsRNA-mediated aggregates in transgenic adult flies and larvae. To identify CR-positive aggregates prior to heat shock and following 1 hour recovery from a 30 minute heat shock, cytological preparations were made from the brain of adult and larvae, and also from salivary glands, imaginal discs, fat body, mucosa, gut and muscles of the larvae from Canton-S, RQT and RQ2 strains. Solitary CR-positive aggregates were only occasionally seen in all examined tissues of heat shock-treated Canton-S and transgenic RQT larvae and non-treated RQ2 larvae. A dramatic increase of the CR-positive aggregate formation was observed in all tissues after heat shock in RQ2 larvae. The most illustrative case was observed in the salivary glands of RQ2 heat shock treated larvae, where inclusions were seen within 30 minutes as a crown around the nuclei (FIG. 6a) and b). Later the aggregates migrated to the cytoplasm where, though less in number, they could be seen 24 hours after heat shock treatment.

Since the Northern blot analysis revealed a very strong expression of RQ11+12 RNA after heat shock treatment in adult flies and in both larval stages tested, we carried out a detailed histological analysis of the brains from 5 days-old males and females of all the experimental groups. Canton-S and non-treated transgenic flies were used as corresponding controls to reveal CR-positive aggregates in the adult fly brains. CR-positive amyloid inclusions were observed in the brain (especially in the central complex), of each heat shock treated RQ2 male, fixed 1 hour after 30 minutes of heat shock treatment (FIGS. 6c and d), but not in adults who had experienced heat shock at larval stages (HS1 or HS2 series). Characteristically, neither non-treated RQ2 males, nor non-treated or heat shock-treated Canton-S and RQT flies had such inclusions.

To determine the functional consequences of RQ11+12 expression on neural morphology, the frontal protocerebral cellular mass from the brains of heat shock-treated and non-treated RQ2 adults was subjected to a more detailed analysis using a complex Giemsa staining.

Giemsa-positive pycnomorphic cells with a high level of basophilic staining were seen in the RQ2 adult brains 1 hour after heat shock treatment. The dying nerve cells, as well as Giemsa-positive needle-like and filamentous structures near them were also observed (FIGS. 6e and f). Similar structures in close proximity to the nerve cells are often observed in post mortem brains of patients with Alzheimer's and Parkinson's diseases, and tauopathies.

No such neural pathology was evident in heat shock-treated or non-treated Canton-S flies, or in RQ2 transgenic flies without heat shock.

Courtship Characteristics

Wild type flies of the control group (Canton-S) quickly detect a female (courtship latency is short) and immediately start to follow her persistently and sing a lot, sometimes nearly continuously. Their movements are precisely directed towards the target, and attempts to copulate are frequent. The resulting mating success of Canton-S males is high and duration of courtship necessary to reach copulation is relatively short. Application of heat shock suppresses singing (sexual motivation is much lower—see SI in Table 1) especially in flies of HS2 and HS groups, although courtship behavior of males does not seriously change except that in the HS2 group the courtship duration is nearly twice as long, the courtship efficiency is somewhat lower, and flies of HS group need much more time to start courtship probably as the result of increased anxiety (at the beginning of an experiment, they often ran actively without any interest in the female).

By contrast, RQ2 males from control (not treated by heat shock) and HS1 groups were very active but sometimes ran aloof of the female, often stopping for a while (resembling akinesia), and rarely attempting to copulate. Their sexual motivation, courtship latency and duration are similar to those of corresponding wild type groups but the courtship efficiency is lower especially in HS1 group. Heat shock treatment given during prepupa stage (HS2 group) leads to hyper-excitation of RQ2 males. They became nervous (increased anxiety), often fussily ran mostly aloof of the female (ataxia), or even dashed aside her in case of accidental contacts (dyskinesia), or conversely, suddenly stopped and stayed absolutely motionless for long periods of time (this time akinesia is much stronger than in controls) or repeatedly preened (displacement activity). Their sexual motivation judging from SI values was only slightly lower than the controls (non-heat shock-treated flies), instead of being suppressed as in wild type flies. For males of the HS group, periodic stupor behavior was especially characteristic and they had much longer courtship latency than that seen for wild type males. Their sexual motivation, contrary to the situation with wild type flies of the corresponding HS group, was nearly equal to that of the controls (also no sign of suppression). Following behavior for most of the RQ2 males for all experimental groups was intermittent; courtship bouts were short and rare which might be indicative either of impaired attention, apathy or of problems with visuo-spatial orientation. Their mating success was low. Some males, especially in HS2 group, did not start courtship at all during the full 10 minutes of the test (Table 1, see NC values).

Behavior of RQT157 males, both in the control and HS groups, was more similar to that of wild type animals, although courtship latency for the HS males practically did not increase relative to the controls.

Some courtship and singing parameters in wild-type (CS) and transgenic flies without (control) and after heat shock application at the larval stage (HS₁), prepupal stage (HS₂) or in 5 days old imago 1 hour before the test (HS). Courtship object—virgin Canton-S females 5 days old.

TABLE 1

| | | | | |
|---|---|---|---|---|
| Group | LPs | CDs | CE % | SI % |

Wild Type

| | | | | |
|---|---|---|---|---|
| control | 23.9 ± 4.3 | 153 ± 28.5 | 90 | 20.1 ± 2.8 |
| | N = 20 | N = 18 | | N = 15 |
| | NC - 0 | in 2 > 600s | | |
| HS₁ | 15.3 ± 3.0 | 98 ± 18.7 | 90 | 16.6 ± 2.1 |
| | N = 20 | N = 18 | | N = 11 |
| | NC - 0 | in 2 > 600s | | |
| HS₂ | 37.4 ± 10.4 | 277 ± 45.4* | 65* | 9.3 ± 0.9* |
| | N = 20 | N = 13 | | N = 20 |
| | NC - 0 | in 7 > 600s | | |
| HS | 77.9 ± 25.0* | 186 ± 31.7 | 79 | 8.4 ± 1.5* |
| | N = 19 | N = 15 | | N = 16 |
| | NC - 0 | in 4 > 600s | | |

RQ2

| | | | | |
|---|---|---|---|---|
| control | 40.9 ± 11.2 | 164 ± 44.1 | 75 | 20.1 ± 2.8 |
| | N = 19 | N = 15 | | N = 11 |
| | NC - 1 | in 4 > 600s | | |
| HS₁ | 92.4 ± 29.5* | 104 ± 18.0 | 50 | 18.3 ± 4.1 |
| | N = 16 | N = 10 | | N = 12 |
| | NC - 4 | in 6 > 600s | | |
| HS₂ | 28.3 ± 8.1 | 238 ± 45.4 | 45* | 15.6 ± 3.1 |
| | N = 11 | N = 9 | | N = 12 |
| | NC - 9 | in 2 > 600s | | |
| HS | 72.3 ± 21.3 | 179 ± 31.7 | 65 | 17.7 ± 3.0 |
| | N = 17 | N = 13 | | N = 12 |
| | NC - 3 | in 4 > 600s | | |

RQT157

| | | | | |
|---|---|---|---|---|
| control | 43.0 ± 14.0 | 193 ± 56.8 | 69 | 19.7 ± 2.7 |
| | N = 13 | N = 9 | | N = 12 |
| | NC - 0 | in 4 > 600s | | |
| HS | 56.9 ± 9.0 | 260 ± 38.9 | 90 | 8.3 ± 2.6* |
| | N = 20 | N = 18 | | N = 12 |
| | NC - 0 | in 2 > 600s | | |

LP—courtship latency;
CD—courtship duration before copulation;
CE—courtship efficiency (% of copulated pairs);
SI—singing index.
N—number of measurements,
NC—number of males which did not start to court during the test.
*significant difference from the control (p < 0.05,
Fisher's test for comparison of parts in the case of CE and unpaired t-test for the rest of parameters).

Learning and Memory Under Conditioned Courtship Suppression Paradigm

At present, the conditioned courtship suppression paradigm (CCSP) is widely used for learning ability and memory retention in Drosophila (N G Kamyshev, et al., 1999 Learning and Memory 6: 1-20; R W Siegel, et al., 1979 Proc Natl Acad Sci USA 76:3430-3434).

Although the dynamics of memory retention is usually evident from the analysis of courtship indices (CI) for a given strain, the learning indices (LI) were also computed to enable comparisons to be made irrespective of genotype- or heat shock-introduced fluctuations in the courtship levels of naive males.

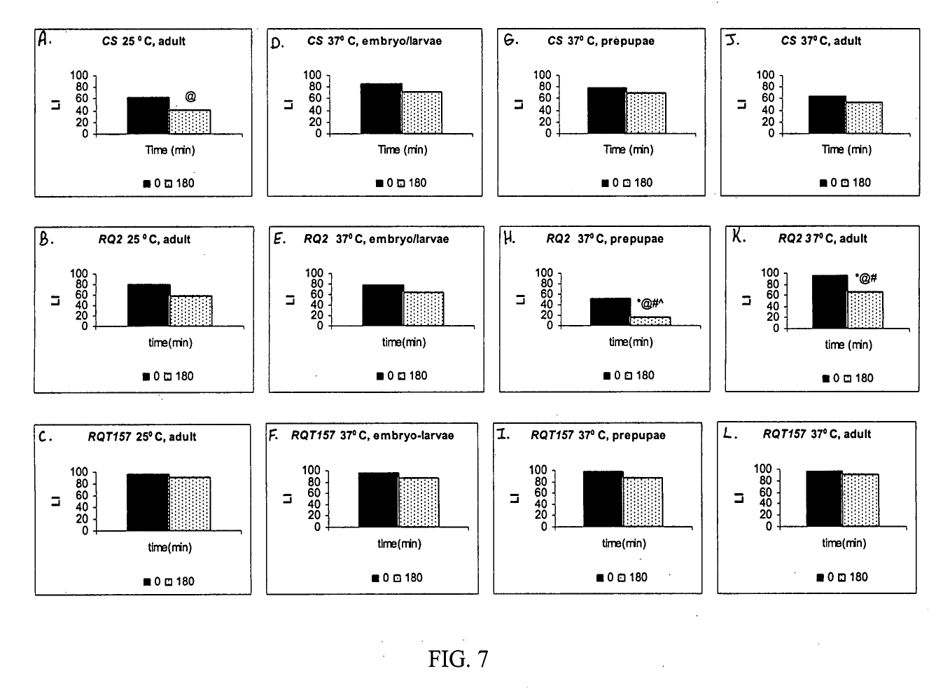

FIG. 7 presents the results of such analyses. Canton-S flies showed a tendency for improved memory formation after experiencing heat shock—there was no time-dependent decline in LI (0 hours vs. 3 hours after training). By contrast, a profound decline in LI 3 hours after training was evident in RQ2, showing a dramatic 3.5-fold decrease in memory formation manifested by males that experienced heat shock at the prepupal stage. These males were also defective in learning acquisition as compared to RQT males.

At the same time RQT males demonstrated perfect learning and memory formation both at normal temperature and after heat shock.

Discussion

The development of animal models for neurodegenerative diseases is of great importance in order to test substitutive or neuroprotective strategies. An animal model should be designed so as to reproduce the full spectrum of disease manifestations, at least the most prominent among them. Unfortunately, many previously studied models of neurodegenerative diseases develop at most as environmentally induced sporadic disorders and their trigger is unknown (H Braak, H 2005 in: “Melvin Yahr Lecture: Parkinsonism and Related Disorders” 11 (Suppl 2): 53). For these reasons, we made an attempt to develop a Drosophila model that would exhibit the most prominent disease manifestations induced by an artificial, species-unspecific and, therefore, possibly a universal trigger for many systemic and neurodegenerative disorders, termed “protein-misfolding disorders”, or more appropriately “protein-conformational disorders” which are characterized by the accumulation of intra- or intercellular protein aggregates.

The shsRNAs described herein can be viewed as a powerful and unique tool to investigate amyloid protein aggregate formation. Previously we used these RNAs to model protein misfolding and aggregation in an in vitro system (B Zeiler, et al., 2003 Biotechnol Appl Biochem 37:173-182; V Adler, et al., 2003 Mol Biol 332: 47-53). The present work describes the effects of expression of these RNAs on protein aggregation in live organisms, the pathological consequences of these aggregates at the histological level, and the effects on sexual motivation, locomotion during courtship behavior, and learning. In contrast to mammalian, fish or C. elegans models which employ chemical lesions (e.g. MPTP, 6-OHDA) or transgenic over expression of a single gene from a subset of genes involved in a polygenic disorder (for review see: N M Bonini and M E Fortini 2003 Ann Rev Neurosci 26:627-656), over expression of shsRNA does not produce any additional, foreign proteins in the model animals since these RNAs do not code for any known proteins. The RNA-modified model animals described herein represent a new type of amyloid disease animal system that can be utilized to model the most common manifestations of various protein-misfolding disorders irrespective of their origin and the major site of the brain lesioning.

The universal nature of the chosen trigger was confirmed by gel-shift assays that demonstrate that shsRQ11+12 RNA can interact with endogenous proteins from Drosophila brain homogenates as it does in humans, mouse, rat and hamster (V Adler, et al., 2003 Mol Biol 332: 47-53). Heat shock-driven over expression of RQ11+12 RNA in transgenic flies leads to cytoplasmic aggregate formation revealed by Congo red staining, pathology of neural cells, altered locomotor display and memory defects, whereas RQT157 transgenic flies in all these respects are similar to wild type flies. This clearly indicates that the specific structure of the RNA stem plays a crucial role in triggering the cascade of aggregate formation and neural dysfunction resulting in defective behavioral display. The hallmark of systemic and neurodegenerative disorders is the formation of inclusion bodies—fibrillar, amyloid-like structures with the ability to bind lipophilic dyes such as Congo red (CR) (C M Dobson 2003 Nature 426: 884-890). We observed CR-positive inclusions in different larval tissues 1 hour after heat shock in RQ11+12 transgenic strains, but only occasionally in RQT or Canton-S wild type strains (see Results and FIG. 6). In general, the aggregate localization in the larval tissues is similar to the localization of the polyQ aggregates found in a Drosophila model of Huntington's disease (C M Dobson 2003 Nature 426: 884-890).

In our investigation we used heat shock treatment to induce the expression of two different shsRNAs, however, it is known that HSP70 might have a protective role in Parkinson's disease and other neurodegenerative diseases (P K Auluck, et al., 2002 Science 295: 865-868). Therefore, the expression of HSP70 and other heat shock proteins may mask the deleterious effects of RQ11+12 RNA over expression induced by heat shock. The main conclusion derived after administrating our approach to the analysis of the whole scope of the data obtained in the present study is: when heat shock is given to induce the expression of a gene under a heat shock promoter, the possible protective role of HSP70 could not overcome the impairments produced by the RQ11+12 RNA over-expression.

The applied scheme of heat shock administration at crucial developmental stages and in adults enables the assessment of immediate and remote effects of heat shockper se, or heat shock-driven induction of a transgene. Heat shock treatment, given at specific developmental stages coinciding with the formation of the brain structures known to be involved in learning, memory storage and control of locomotion, provides the ability to monitor the probable contribution of a structure to behavioral phenotypes observed in wild type flies. On the contrary, for the RQ transgenic strains, due to over-expression of the RQ11+12 RNA we score the consequences of the neural dysfunction produced by a lesioned neural circuits of a certain brain structure probably due to aggregate formation and, presumably, by the death of lesioned neurons. Hence, we compare the consequences of “heat shock modulation” vs. “heat shock-induced shsRNA lesioning”.

Although we can observe aggregate formation in a number of tissues of RQ11+12-containing transgenic strains after heat shock irrespective of the stage of heat shock administration, the remote effects of developmental heat shock (HS1 and HS2 series) are absent at the level of the histological inspection of the brain in adults, probably because lesioned neurons and aggregates per se do not pass through metamorphosis. However, the remote effects of heat shock treatment are manifested at the behavioral level, leading to alterations of sexual motivation, courtship behavior, locomotion and memory formation described above in the Results section. One can ascribe these phenomena to deleterious effects of formation of cytoplasmic amyloid aggregates formed immediately after heat shock and scattered over the central complex, the optic lobes and the fronto-lateral protocerebral cellular mass. It is known that the central complex has polysynaptic connections both with ascending interneurons of different motor and sensory systems and with descending interneurons, controlling activity of motor centers during realization of different behavioral acts (R Wolf, et al., 1998 Learning and Memory 5:166-178; A Popov, et al., 2004 Zh. Evol. Biochem. Fiziol. 40: 521-530). What might be the physiological consequence of aggregate formation? As demonstrated in a Drosophila model of Huntington's disease (C M Dobson 2003 Nature 426: 884-890), the cytoplasmic aggregate formation sequesters endogenous polyglutamine proteins and blocks axonal transport, contributing to neurophysiological dysfunction presumably due to synaptic pathology. For example, in human brain, a widespread loss of synapses, up to 50% in some patients, occurs during Alzheimer's disease, whereas neuronal loss is more selective and localized (E Masliah, et al., 1989 Neurosci Lett 103: 234-239). On the other hand crucial evidence about the role of protein aggregates in neural pathology indicate that: (a) the specific neuronal pathology precedes the appearance of protein aggregates in mouse models of disease; (b) the neurodegenerative disease in patients occurs with comparable severity when visible protein aggregates are absent; and (c) the neurotoxicity in vitro is best reproduced by oligomers, not polymers of the mutant disease protein. It is also feasible that protein aggregates are inert byproducts of the disease protein malconformation, or that they even represent beneficial cellular efforts to sequestrate the soluble toxic disease protein, together with normal or aberrant interactor proteins. Importantly, not only the CR-positive aggregates, but also the dying nerve cells, as well as the Giemsa-positive, needle-like- and filamentous structures near to the dying cells were present in the fronto-lateral protocerebral cellular mass in the brain of RQ2 transgenic strain 1 hour after heat shock treatment (FIGS. 6e and f). Similar structures found in close proximity to nerve cells are often observed in post mortem brains of Alzheimer patients.

In summary, the RQ11+12 transgenic flies are an appropriate model for neurodegenerative, protein-folding diseases, since the model recapitulates the main disease manifestations—motor and memory impairments, and aggregate formation. It closely resembles the typical cognitive profile of dementia in Parkinson's disease—a dysexecutive syndrome with impaired and fluctuating attention, apathy (low motivation), and prominent impairment of executive and visuo-spatial functions (M Emre 2005 Parkinsonism and Related Disorders 11 (Suppl 2): 64).

Which proteins are involved in these aberrant protein interactions, how do they mediate neuronal dysfunction and what is the role of molecular chaperones in this effect? Ultimately, molecular genetic approaches will be required to enable us to answer these questions. Our model seems to be promising in this respect.

The results provided herein indicate that expression of RQ11+12 in Drosophila induces amyloid-like aggregate formation in the brain of adult transgenic flies and larvae as well as in various larvae tissues. Aggregate formation in the RQ11+12 transgenic flies was found to correlate with impaired learning acquisition and memory retention in adult males and to a lesser extent with a decline in the efficiency of their mating behavior due to increased time spent in non-courtship activities. Transgenic flies generated with RQT157 RNA were similar to control flies (non-transgenic, Canton-S flies) for most of the parameters that were evaluated.

These results suggest that constructed transgenic RQ 11+12 Drosophila are a useful model for the study of molecular biology of the misfolded protein disorders.

### Example 2

### Transforming Drosophila Melanogaster Cells

The vector was injected by microinjection techniques into Drosophila melanogaster eggs. This process is depicted in FIG. 2. The eggs were incubated and Drosophila exhibiting white eyes are identified. These Drosophila were allowed to reproduce. Eggs from such Drosophila are divided into control and experimental groups. The experimental groups are subjected to heat shock during different stages of development. The control groups were not subjected to heat shock.

### Example 3

### Monitoring Transformed Drosophila Melanogaster for Behavior Changes

Drosophila melanogaster testing is performed after a period has passed for the non-coding RNA to cause an effect. Five days is generally sufficient. The transformed Drosophila expressing the non-coding RNA exhibit several behavior abnormalities. The Drosophila melanogaster expressing the non-coding RNA exhibit impaired learning ability to differentiate receptive female flies, reduced memory and incorrect mating sounds.

### Example 4

### Evaluation of Tissues for Agglomeration

Expression the non-coding RNA is confirmed by Northern blot in the manner known in the art. Brain tissue from Drosophila melanogaster is obtained from control flies and experimental flies. The flies expressing the non-coding RNA exhibit plaque formation in neuronal structures of the brain. Amyloid deposits are highlighted with Congo red stain revealed under photomicroscope. Other tissues are evaluated in a similar manner for agglomerations.

### Example 5

### Identification of Drug Candidates

Transformed Drosophila melanogaster are placed in two groups. One group is subjected to potential drug candidates by placing such candidate in water and food stuff. The other is a control. In the event the flies of the experimental group exhibit fewer pathologies or impairments, such group may have been subjected to a composition useful as a drug for the treatment of agglomeration disease.

### Example 6

### Construction of Trangene for Mouse

A plasmid would be modified to incorporate a DNA construct comprising a mammalian promoter, the DNA for RQ 11+12, in a manner known in the art. This vector is multiplied in E. coli and harvested in the manner known to individuals skillful in art.

### Example 7

### Transforming Mouse Cells

The vector would be injected by microinjection techniques into the pronucleus one day male mouse embryos. The eggs would be implanted into the oviduct of psuedopregnant foster female mice. The transformed mice embryos are matured into pups and born. Mice exhibiting the genotype are identified by Southern Blot analysis of the mice tissue. These mice would be allowed to reproduce.

### Example 8

### Monitoring Transformed Mouse for Behavior Changes

The transformed mice would be monitored for behavior changes. The behavior changes would comprise memory loss and other mental impairment.

### Example 9

### Evaluation of Tissues for Agglomeration

Tissues from the transformed mice would be evaluated. The tissue would be examined for plaque formation in brain tissue of the type found in spongiform encephalopathies.

### Example 10

### Identification of Drug Candidates

Mice exhibiting features of agglomeration disease would be subjected to drug candidates. Compositions which altered the progression of the disease compared to control animals, would be drug candidates.

Thus, the present invention has been described in detail with the understanding that such detailed description is directed to preferred embodiments of the present invention. The present description is subject modification and alteration, which modification and alteration should include the subject matter and its equivalents of the claims that follow.

We claim:

1. A non-human transgenic animal for use in the study of diseases associated with protein misfolding, wherein said transgenic animal expresses an untranslated non-coding RNA capable of binding an agglomeration protein and mediating a conformational change in said agglomeration protein.

2. The transgenic animal of claim 1, wherein said transgenic animal is Drosophila melanogaster.

3. The transgenic animal of claim 1, wherein said animal is a mouse.

4. The transgenic animal of claim 1, wherein said RNA has a sequence having at least one section of three or more nucleotides that are recognized by a RNA dependent RNA polymerase.

5. The transgenic animal of claim 4, wherein said RNA is RQ 11+12.

6. The transgenic animal of claim 1, wherein said disease associated with protein misfolding is Alzheimer's disease.

7. The transgenic animal of claim 1, wherein said agglomeration protein is an amyloid protein.

8. The transgenic animal of claim 7, wherein said amyloid protein is a tau protein or a β-amyloid protein.

9. A method of evaluating compositions for participation in diseases associated with protein misfolding comprising the steps of:
(a) providing a non-human transgenic animal, wherein said transgenic animal expresses an untranslated non-coding RNA capable of binding an agglomeration protein and mediating a conformational change in said agglomeration protein; and
(b) placing the composition to be evaluated in said transgenic animal and evaluating the animal for at least one characteristic of disease associated with protein misfolding.

10. The method of claim 9, wherein said transgenic animal is Drosophila melanogaster.

11. The method of claim 9, wherein said transgenic animal is a mouse.

12. The method of claim 9, wherein said RNA has sequences derived from retrovirus.

13. The method of claim 12, wherein said RNA sequence derived from retrovirus is HIV.

14. The method of claim 9, wherein said RNA has a sequence having at least one section of three or more nucleotides that are recognized by a RNA dependent RNA polymerase.

15. The method of claim 14, wherein said RNA is RQ 11+12.

16. The method of claim 9, wherein said characteristic is selected form the group consisting of behavior abnormalities and plaque formation.

17. The method of claim 16 wherein said plaque formation is in neural tissue.

18. A method of making a non-human transgenic animal expressing characteristics of a disease associated with protein misfolding comprising the steps of:
(i) inserting a nucleic acid molecule in an animal cell, wherein the nucleic acid molecule further comprises a promoter joined to a nucleic acid sequence encoding for an RNA capable of binding an agglomeration protein and mediating a conformational change in said agglomeration protein under the control of a promoter, thereby resulting in a non-human transgenic animal cell; and
(ii) growing a whole animal from the animal cell, thereby resulting in a non-human transgenic animal expressing the characteristics of a disease associated with protein misfolding.
